(19) United States
(12) Patent Application Publication
(10) Pub. No.: US 2018/0035940 A1
(43) Pub. Date: Feb. 8, 2018

(54) SWEAT TESTING SYSTEM

(71) Applicant: St. Vincent Hospital and Health Care Center, Inc., Indianapolis, IN (US)

(72) Inventors: Ralph Reiff, Indianapolis, IN (US); Lindsay Langford, Indianapolis, IN (US)

(21) Appl. No.: 15/666,998

(22) Filed: Aug. 2, 2017

Related U.S. Application Data
(60) Provisional application No. 62/370,034, filed on Aug. 2, 2016.

(51) Int. Cl. A61B 5/00; A61B 5/11

(52) U.S. Cl. CPC A61B 5/4266; A61B 5/0024; A61B 5/1112; A61B 5/742; A61B 5/486; A61B 2560/0247; A61B 2503/10

(57) ABSTRACT

A system may be configured to provide body performance outputs in response to body performance inputs. The system may include a user interface, a processor, and a memory having a program communicatively connected to the processor. The processor may provide operations to receive a historical excretion rate of a user, receive a body parameter of the user, receive a sensor output associated with the user, generate a projected excretion rate of the user based on the historical excretion rate, the body parameter, and the sensor output, and display by the user interface a body performance output of the user in response to the same.

### CROSS-REFERENCE TO RELATED APPLICATIONS

This Non-Provisional Application is based on and claims priority to US Provisional Patent Application No. 62/370,034 filed Aug. 2, 2016, which hereby incorporated by reference in its entirety.

### BACKGROUND

Traditional sweat testing is performed in a laboratory environment that measures sweat content and sweat rate of an athlete to understand past hydration and nutritional demands of the athlete. Not only are typical tests time consumptive and inconvenient for athletes, but the bodily and environmental circumstances may drastically change between the testing and the athletic performance. Traditional tests are further hindered by high costs and lack of knowledgeable providers and market acceptance. In addition, athletic performance under a set of conditions or at a certain geographical location or elevation may differ when being performed in a different situation.

There is a need for a system that addresses the shortcomings of traditional sweat testing systems. An exemplary sweat testing system that may generate and communicate body performance information such as sweat testing information. Further, a sweat testing system may be utilized by a user such as an athlete and in any environment. A sweat testing system may also provide body performance outputs indicating replacement demands of the user, e.g., in real time. Thus, the sweat testing system may provide user-specific outputs with improved speed and accuracy thereby optimizing athletic performance of the user.

### BRIEF DESCRIPTION OF THE DRAWINGS

FIG. 1 illustrates an exemplary system of the present disclosure;

FIG. 2 illustrates an exemplary user interface of the present disclosure;

FIG. 3 illustrates another exemplary user interface;

FIG. 4 illustrates another exemplary user interface;

FIG. 5 illustrates another exemplary user interface;

FIG. 6 illustrates another exemplary user interface;

FIG. 7 illustrates another exemplary user interface;

FIG. 8 illustrates an exemplary method of the present disclosure;

FIG. 9 illustrates an exemplary user interface including, for example, a pre-activity operation;

FIG. 10 illustrates another exemplary user interface including, for example, a pre-activity operation;

FIG. 11 illustrates another exemplary user interface including, for example, a pre-activity operation;

FIG. 12 illustrates another exemplary user interface including, for example, an activity operation;

FIG. 13 illustrates another exemplary user interface including, for example, a post-activity operation;

FIG. 14 illustrates another exemplary user interface including, for example, activity results;

FIG. 15 illustrates another exemplary user interface including, for example, activity results;

FIG. 16 illustrates another exemplary user interface including, for example, activity results;

FIG. 17 illustrates another exemplary user interface having input selections for generating a plan;

FIG. 18 illustrates another exemplary user interface showing aspects of the plan;

FIG. 19 illustrates another exemplary user interface showing aspects of the plan;

FIG. 20 illustrates another exemplary user interface showing aspects of the plan;

FIG. 21 illustrates another exemplary user interface showing aspects of the plan;

FIG. 22 illustrates another exemplary user interface showing access to past plans; and

FIG. 23 illustrates an exemplary process of the present disclosure.

### DETAILED DESCRIPTION

A system may be configured to provide body performance outputs in response to body performance inputs. The system may include a user interface, a processor, and a memory having a program communicatively connected to the processor. The processor may provide operations to receive a historical excretion rate (e.g., sweat rate) of a user, receive a sensor output (e.g., body sensor output), generate a projected excretion rate (e.g., sweat rate) of the user based on the historical excretion rate and the sensor output, and display by the user interface a body performance output of the user. Further, a non-transitory computer-readable medium tangibly embodying computer-executable instructions of a program may be executable by a processor of a computing device to provide operations to receive a historical excretion rate of a user, receive a sensor output, generate a projected excretion rate of the user based on the historical excretion rate and the sensor output, and display a body performance output of the user. Moreover, a method may comprise receiving a historical excretion rate of a user, receiving sensor outputs, generating a projected excretion rate of the user based on the historical excretion rate and the sensor outputs, and displaying a body performance output of the user.

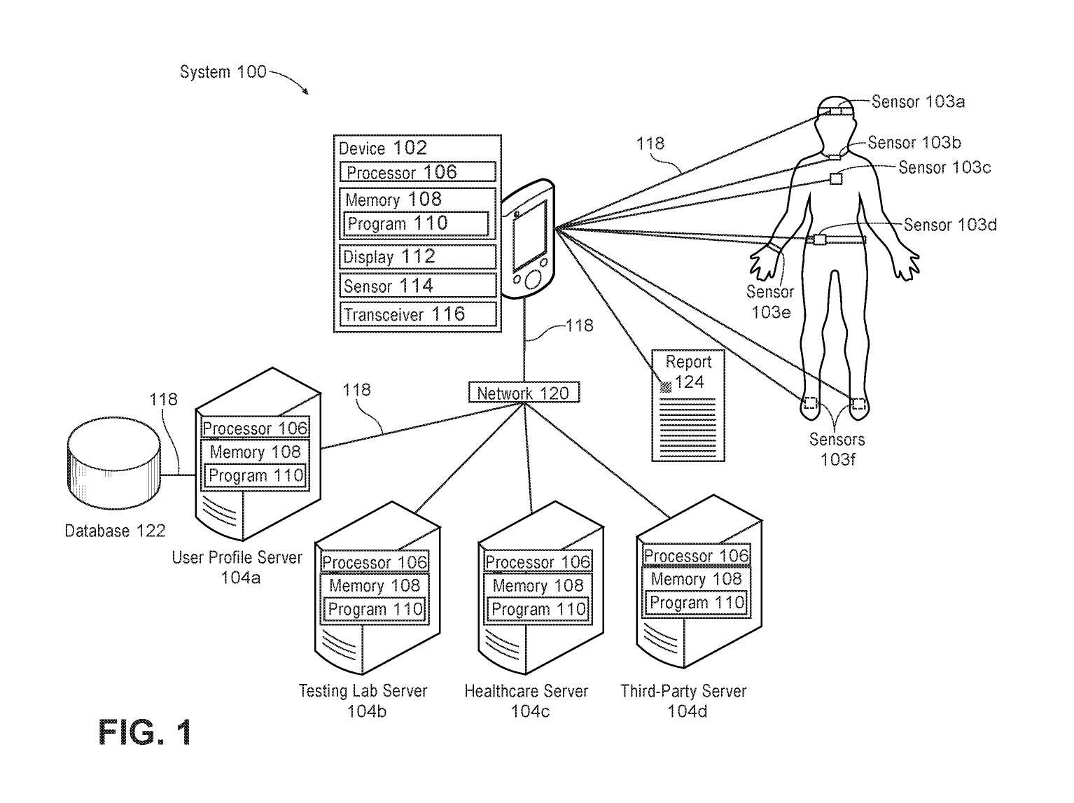

FIG. 1 illustrates an exemplary system 100, for example, to generate and communicate body performance information, e.g., sweat testing information of a user such as an athlete. System 100 may include a device 102, sensors 103a-f, servers 104a-d, processor 106, memory 108, program 110, user interface or display 112, sensor 114, transceiver 116, connection 118, network 120, database 122, and report 124. System 100 may take many different forms and include multiple and/or alternate hardware components and implementations. While an exemplary system 100 is shown in FIG. 1, the exemplary components illustrated in FIG. 1 are not intended to be limiting. Indeed, additional or alternative components and/or implementations may be used.

Body performance information may include any inputs to and outputs from the systems, devices, and operations of the present disclosure. Body performance information may include an excretion rate or sweat rate, which may be used interchangeably. Body performance information may include body performance inputs received by device 102 and body performance outputs generated by device 102. Device 102 may receive body performance information with display 112 or transceiver 116, generate body performance information with processor 106 executing program 110, store body performance information with memory 108, display body performance information with display 112, and communicate body performance information with transceiver 116.

Body performance inputs may include, for example, a historical excretion or sweat rate. The historical excretion rate may include the weight or volume of fluid including sweat and electrolytes that are excreted or otherwise lost by a user during a historical testing period. The body performance inputs may be stored on memory 108, stored on database 122, printed on report 124, or a combination thereof. The body performance inputs may be received by device 102 using sensor outputs of sensors 103a-f or 114, machine readable or textual information from report 124, data stored on memory 108 or database 122, or a combination thereof. The device 102 may include program 110 stored on memory 108 and that when executed by processor 106 receives body performance inputs and generates body performance outputs in response to the body performance inputs. Excretion or sweat rate may also be used interchangeability with excretion or sweat amount or excretion or sweat factor.

Body performance outputs may include a projected excretion or sweat rate and a replacement rate of one or more users. The projected excretion rate may include a projected weight or fluid of fluid such as sweat that is expected to be excreted by the user during a projected performance period. The projected excretion rate may be based on the historical excretion rate of the user or other users, body parameters (e.g., weight, age, and medical history) of the user or comparable users, environmental parameters (e.g., user location, altitude, or weather) of the user or comparable users, or a combination thereof. The replacement rate may include nutritional requirements (e.g., sodium, potassium, carbohydrates, and fluid replacement rates) of the user based on the historical excretion rate and the projected excretion rate. For example, the sodium, potassium, and fluid replacement rates may be matched by fluid volume of the excretion rate according to a range of 40 to 60 percent (e.g., about 50 percent) for mild temperatures (e.g., at or below 50 degrees Fahrenheit), a range of 50 to 70 percent (e.g., about 60 percent) for moderate temperatures (e.g., at or between about 50 to 90 degrees Fahrenheit), and a range of 60 to 80 percent (e.g., about 70 percent) for hot temperatures (e.g., at or above 90 degrees Fahrenheit). Carbohydrate replacement rates may be in a range of 30 to 60 grams of carbohydrates per hour for a user of an average body weight, and with the range being higher for a higher body weight and lower for a lower body weight.

Any or all of sensors 103a-f may include a body sensor configured to detect and generate one or more sensor outputs associated with the body of the user. Sensors 103a-f may include any sensor configured to measure and monitor one or more body parameters of the user and generate sensor outputs including body performance information. For example, sensor 103a may be positioned on a forehead of a user or connected to a headband, sensor 103b may be positioned near a neck of the user or connected to a necklace, sensor 103c may be positioned near a chest of the user or connected to clothing, sensor 103d may be positioned near a waist of the user or connected to a belt or waistband, sensor 103e may be positioned near an arm, hand, or wrist or may connected to a bracelet, and sensor 103f may be positioned near a leg, foot, or ankle or connected to a shoe.

Sensors 103a-f may include any wired or wireless sensor, for example, any health monitor or any wearable, contact, or non-contact sensor. Sensors 103a-f may be unattached, releasably attached, or permanently attached with respect to the user or accessories of the user (e.g., clothing, shoes, glasses, or jewelry). An of sensors 103a-f may include a heart rate monitor, cardiac sensor, blood glucose meter, respirometer, spirometer, respiration sensor, optical sensor, electrocardiogram (EKG), medical imaging device, medical radar device, pulse oximeter, blood pressure monitor, body temperature monitor, breathalyzer, chemical sensor, or moisture sensor, as examples. The sensor outputs may include body parameters such as a heart rate, glucose level, respiratory rate, eye response, oxygen saturation, blood pressure, body temperature, or a combination thereof. Sensors 103a-f may be configured to communicate one or more sensor outputs (e.g., real-time, near real-time, periodically, or upon request of a user) to the device 102, which may communicate body performance information, the sensor outputs, or a combination thereof to any or all of display 112 and servers 104a-d.

Any or all of sensors 103a-f and 114 may include an environmental sensor, a microphone, camera, scanner, or a combination thereof. Sensors 103a-f and 114 may be communicatively connected to or part of device 102. Sensors 103a-f and 114, using transceiver 116, may be configured to communicate a sensor output (e.g., real-time, near real-time, periodically, or upon request of the user) to any or all of display 112 and servers 104a-d.

In use, device 102 may receive (e.g., using display 112 as a user interface) body performance information from the user, receive (using transceiver 116 by way of network 120) body performance information from one or more of sensors 103a-f and 114 and servers 104, display (e.g., with display 112) body performance information, communicate (e.g., using the transceiver 116 by way of network 120) the body performance information to any of servers 104a-d, and cause storage of body performance information on database 122.

System 100 may include an overall network infrastructure through which the device 102, sensors 103a-f, servers 104a-d, and database 122 may communicate, for example, to transfer body performance information between any of device 102, sensors 103a-f, servers 104a-d, and database 122, e.g., using connections 118. In general, a network (e.g., system 100 or network 120) may be a collection of computing devices and other hardware to provide connections and carry communications.

Device 102 may include any computing device such as include a mobile device, cellular phone, smartphone, smartwatch, activity tracker, tablet computer, next generation portable device, handheld computer, notebook, or laptop. Device 102 may include processor 106 that executes program 110. Device 102 may include memory 108 that stores body performance information and program 110. Device 102 may include transceiver 116 that communicates body performance information between any of device 102, sensors 103a-f, servers 104a-d, and database 122.

Servers 104a-d may include any computing system. For example, server 104a may include a user profile server for generating and storing a user profile for each user, server 104b may include a testing lab server for generating and storing report 124, server 104c may include a healthcare server for generating and storing a medical history of each user, and server 104b may include a third-party server for generating and storing body performance information related to other users and devices. Servers 104a-d may be configured to communicatively connect with and transfer information between each other and with respect to the device 102, and database 122. Servers 104a-d may be in continuous or periodic communication with device 102. Servers 104a-d may include a local, remote, or cloud-based server or a combination thereof and may be in communication with and provide body performance information (e.g., as part of memory 108 or database 122) to any of device 102. Servers 104a-d may further provide a web-based user interface (e.g., an internet portal) to be displayed by display 112. Servers 104a-d may communicate the body performance information with device 102 using a notification including, for example automated phone call, short message service (SMS) or text message, e-mail, http link, web-based portal, or any other type of electronic communication. In addition, servers 104a-d may be configured to store body performance information as part of memory 108 or database 122. Servers 104a-d may include a single or a plurality of centrally or geographically distributed servers 104. Servers 104a-d may be configured to store and coordinate body performance information with device 102 and database 122.

Display 112 of device 102 may include any hardware such as a display, screen, user interface, or mechanism to connect to a display, support user interfaces, and communicate body performance information within the system 100. Any of the inputs into and outputs from display 112 may be included into body performance information. Display 112 may include any input or output device to facilitate the receipt or presentation of information (e.g., body performance information) in audio, visual or tactile form or a combination thereof. Examples of a display may include, without limitation, a touchscreen, cathode ray tube display, light-emitting diode display, electroluminescent display, electronic paper, plasma display panel, liquid crystal display, high-performance addressing display, thin-film transistor display, organic light-emitting diode display, surface-conduction electron-emitter display, laser TV, carbon nanotubes, quantum dot display, interferometric modulator display, and the like. Display 112 may present user interfaces to any user of device 102.

Device 102 and network 120 may include or utilize location determination technology that enables the determination of location information (e.g., a current geographic position) of the user of device 102. Examples of location determination technology may include or utilize, without limitation, global positioning systems (GPS), indoor positioning system, local positioning system, mobile phone tracking, and cellular triangulation. Device 102 may determine location in conjunction with network 120. Device 102 may be configured to provide a current geographic position of device 102, for example, to provide the user location.

Connections 118 may be any wired or wireless connections between two or more endpoints (e.g., devices or systems), for example, to facilitate transfer of body performance information. Connection 118 may include a local area network, for example, to communicatively connect device 102 with network 120. Connections 118 may include a wide area network connection, for example, to communicatively connect servers 104a-d with network 120. Connection 118 may include a radiofrequency (RF), near field communication (NFC), bluetooth, Wi-Fi, or a wired connection, for example, to communicatively connect the device 102 and sensors 103.

Any portion of system 100, e.g., device 102 and servers 104a-d, may include a computing system and/or device that includes processor 106 and memory 108. Computing systems and/or devices generally include computer-executable instructions, where the instructions may be executable by one or more devices such as those listed below. Computer-executable instructions may be compiled or interpreted from computer programs created using a variety of programming languages and/or technologies, including, without limitation, and either alone or in combination, Java™, C, C++, Visual Basic, Java Script, Perl, SQL, PL/SQL, Shell Scripts, etc. System 100, e.g., device 102 and servers 104a-d may take many different forms and include multiple and/or alternate components and implementations, as illustrated in the Figures further described below. While exemplary systems, devices, modules, and sub-modules are shown in the Figures, the exemplary components illustrated in the Figures are not intended to be limiting. Indeed, additional or alternative components and/or implementations may be used, and thus the above communication operation examples should not be construed as limiting.

In general, computing systems and/or devices (e.g., device 102 and servers 104a-d) may employ any of a number of computer operating systems, including, but by no means limited to, versions and/or varieties of the Microsoft Windows® operating system, the Unix operating system (e.g., the Solaris® operating system distributed by Oracle Corporation of Redwood Shores, Calif.), the AIX UNIX operating system distributed by International Business Machines of Armonk, N.Y., the Linux operating system, the Mac OS X and iOS operating systems distributed by Apple Inc. of Cupertino, Calif., the BlackBerry OS distributed by Research In Motion of Waterloo, Canada, and the Android operating system developed by the Open Handset Alliance. Examples of computing systems and/or devices such as device 102 and servers 104a-d may include, without limitation, mobile devices, cellular phones, smart-phones, super-phones, tablet computers, next generation portable devices, mobile printers, handheld computers, notebooks, laptops, secure voice communication equipment, networking hardware, computer workstations, or any other computing system and/or device.

Further, processors such as processor 106 may include any hardware (e.g., a hardware processor) configured to receive instructions from memories such as memory 108 or database 122 and execute the instructions, thereby performing one or more processes, including one or more of the processes described herein. Such instructions and other body performance information may be stored and transmitted using a variety of computer-readable mediums (e.g., memory 108 or database 122). Processors such as processor 106 may include any computer hardware or combination of computer hardware that is configured to accomplish the purpose of the devices, systems, and processes described herein. For example, processor 106 may be any one of, but not limited to single, dual, triple, or quad core processors (on one single chip), graphics processing units, visual processing units, and virtual processors.

Memories such as memory 108 or database 122 may include, in general, any computer-readable medium (also referred to as a processor-readable medium) that may include any non-transitory (e.g., tangible) medium that participates in providing body performance information or instructions that may be read by a computer (e.g., by the processors 106 of the device 102 and servers 104a-d). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including radio waves, metal wire, fiber optics, and the like, including the wires that comprise a system bus coupled to a processor of a computer. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

Further, databases, data repositories or other body performance information stores (e.g., memory 108 and database 122) described herein may generally include various kinds of mechanisms for storing, providing, accessing, and retrieving various kinds of body performance information, including a hierarchical database, a set of files in a file system, an application database in a proprietary format, a relational database management system (RDBMS), etc. Each such body performance information store may generally be included within (e.g., memory 108) or external (e.g., database 122) to a computing system and/or device (e.g., device 102 and servers 104a-d) employing a computer operating system such as one of those mentioned above, and/or accessed via a network (e.g., system 100 or network 120) or connection in any one or more of a variety of manners. A file system may be accessible from a computer operating system, and may include files stored in various formats. An RDBMS generally employs the Structured Query Language (SQL) in addition to a language for creating, storing, editing, and executing stored procedures, such as the PL/SQL language mentioned above. Memory 108 and database 122 may be connected to or part of any portion of system 100.

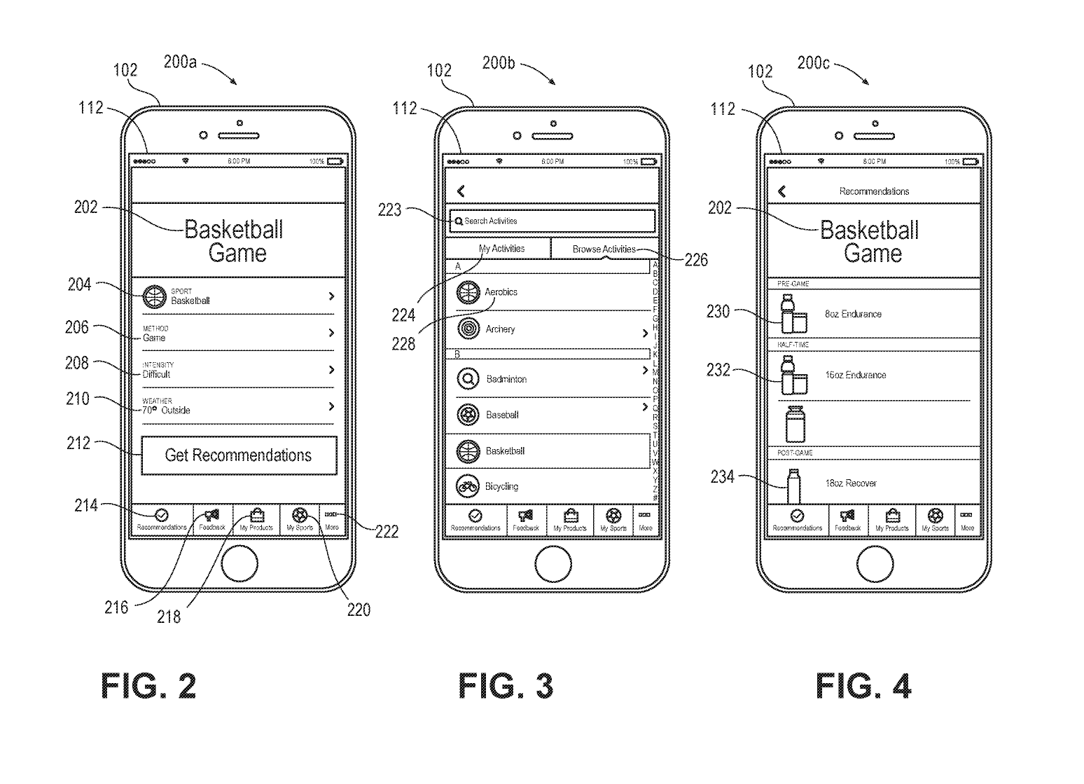

FIGS. 2-4 illustrate an exemplary system 200, e.g., to display body performance information, e.g., sweat testing information of a user such as an athlete. System 200 may take many different forms and include multiple and/or alternate hardware components and implementations. While an exemplary system 300 is shown in the figures, the exemplary components illustrated are not intended to be limiting. Indeed, additional or alternative components and/or implementations may be used.

For instance, FIG. 2 illustrates system 200a having device 102 with user interface or display 112, e.g., for receiving body performance inputs. Display 112 may display an activity name 202 (e.g., basketball game), activity selection 204 (e.g., basketball, aerobics, archery, badminton, baseball, bicycling, running, etc.), method selection 206 (e.g., a game, practice, training, etc.), intensity selection 208 (e.g., difficult, moderate, or easy), weather status 210 (e.g., outside temperature, humidity, and weather condition such as sunny, rain, snow, or thunderstorm), and get or generate recommendations 212. Display 112 may further include recommendations 214 including nutritional recommendations for the user, feedback 216 including feedback for the user in optimizing performance. My product 218 includes nutritional products that are being used by the user, my sports 220 includes activates such as sports selected by the user, and “more” tab 222 includes further selections and setup options. Advancing to the next screen, such as to FIG. 3, may be via a “swipe” on a touchscreen of display 112, or it may be via a “next” button, as described below with respect to additional figures and as discussed with respect to element 510 below.

FIG. 3 illustrates system 200b having device 102 with display 112, e.g., for receiving body performance inputs. Display 112 may display search activities 223, my activities 224, browse activities 226, and activity selections 228, e.g., in response the user selecting the sport selection 204. Search activities 223 may initiate a textual search of prior selections, current selections, and body performance information of the user. My activities 224 may initiate display of prior selections, current selections, and body performance information of the user. Browse activities 226 may cause the display of activity selections 228 to allow the user to select or deselect activities (e.g., aerobics, archery, badminton, baseball, basketball, bicycling, running, etc.).

FIG. 4 illustrates system 200c having device 102 with display 112, e.g., for displaying body performance outputs in response to the body performance inputs. Display 112 displays an activity name (202) and may include a pre-activity recommendation 230 including nutritional recommendations prior to an activity such as a game (e.g., a first listing of beverage, food, or supplement items), a during activity recommendation 232 including nutritional recommendations during the activity (e.g., a second or updated listing of beverage, food, or supplement items), and a post-activity recommendation 234 including nutritional recommendations after the activity (e.g., a third or further updated listing of beverage, food, or supplement items).

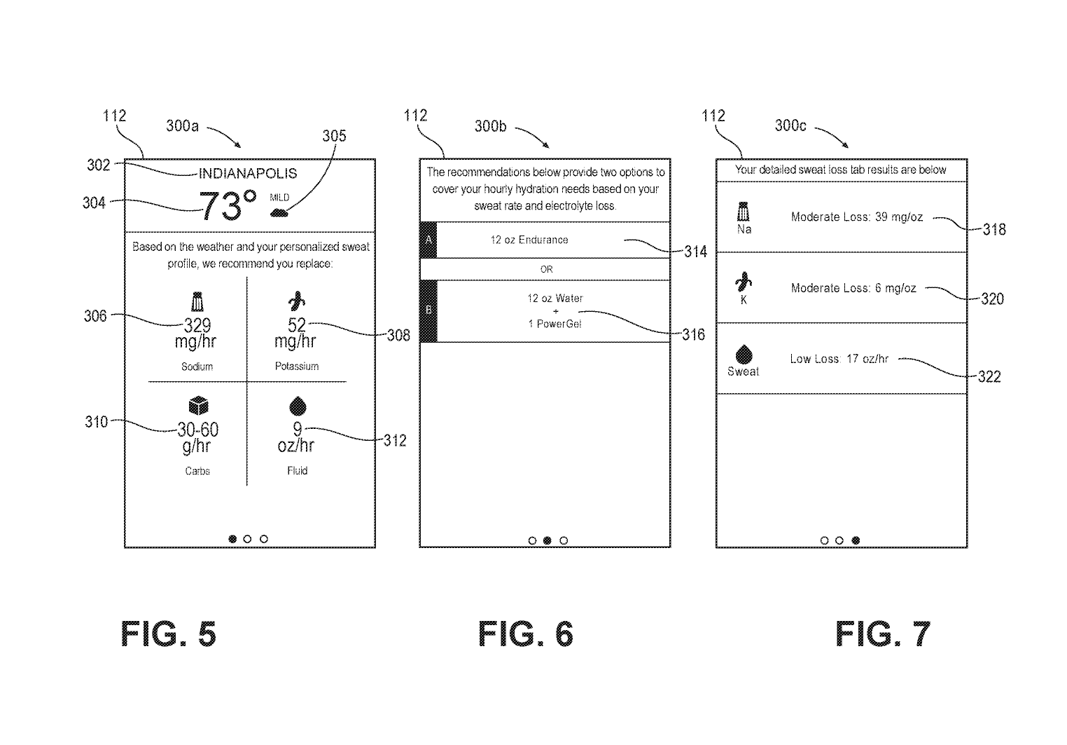

FIGS. 5-7 illustrate an exemplary system 300a-c, e.g., to display body performance information, e.g., sweat testing information of a user such as an athlete. System 300a-c may take many different forms and include multiple and/or alternate hardware components and implementations. While an exemplary system 300a-c is shown in the figures, the exemplary components illustrated are not intended to be limiting. Indeed, additional or alternative components and/or implementations may be used.

FIG. 5 illustrates system 300a with display 112, e.g., for displaying body performance outputs in response to body performance inputs. Display 112 may display environmental parameters such as a user geographic location 302 and an environmental temperature 304. Display 112 may further display replacement rates including a sodium replacement rate 306, a potassium replacement rate 308, a carbohydrate replacement rate 310, and a fluid replacement rates 312, e.g., based on a historical excretion rate, a projected excretion rate, or a combination thereof.

FIG. 6 illustrates system 300b with display 112, e.g., for displaying performance outputs in response to performance inputs. Display 112 may include a single recommendation 314 (e.g., one beverage, food, or supplement item) or a combination recommendation 316 (e.g., a combination of beverage, food, or supplement items). Recommended beverages, or combination of beverages, may be based on one, or a combination, of the rates of depletion or reduction of the items identified in FIG. 5, depending on which activity is performed, its duration, and its level.

FIG. 7 illustrates system 300c with display 112, e.g., for displaying performance outputs corresponding to performance inputs such as from report 124. Display 112 may display a loss level (e.g., low, mild, or high) for each body parameter. Display 112 may display a sodium loss level 318, a potassium loss level 320, and a sweat or fluid loss level 322 (which may correlate with an amount of mass, such as sodium (Na), potassium (K), per mass of the user body weight in units of mg/ounce, or sweat in units of ounces/hour). And, although units of ounce are used for the mass of sweat, it is contemplated that any unit of mass may be used, such as milligrams or grams.

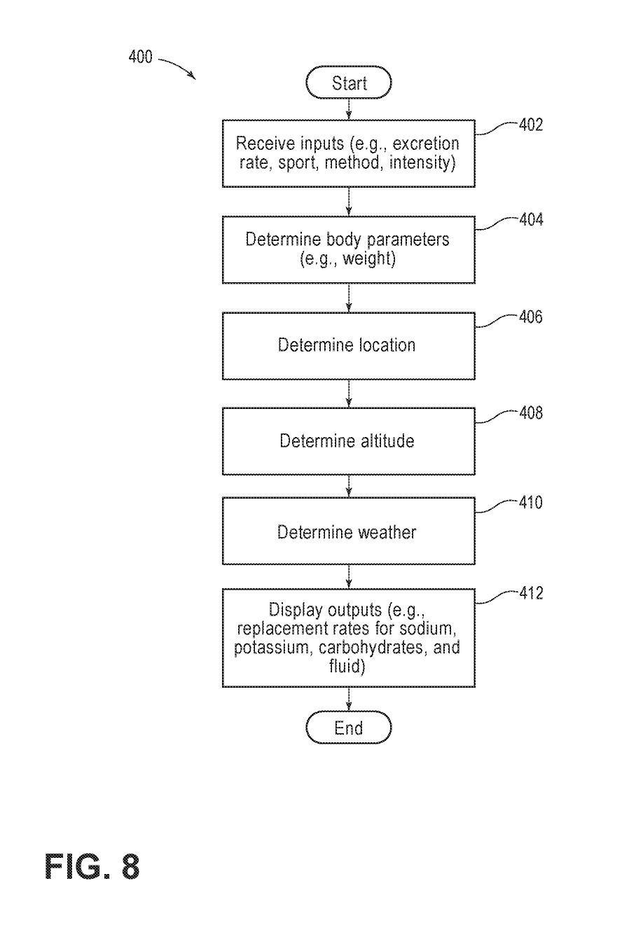

FIG. 8 illustrates an exemplary process 400. Process 400 may take many different forms and include multiple and/or alternate components and implementations. While an exemplary process 400 is shown in FIG. 8, the exemplary components illustrated in FIG. 8 are not intended to be limiting. Indeed, additional or alternative components and/or implementations may be used.

At block 402, device 102, or any of servers 104, may receive inputs (e.g., exertion rate, sport, method, and intensity) by way of sensors 103 or 114, display 112, network 120, or any combination thereof.

At block 404, device 102, or any of servers 104, may determine body parameters by way of display 112, network 120, or a combination thereof.

At block 406, device 102, or any of servers 104, may determine a first environmental parameter, e.g., a geographic location of the user or device 102.

At block 408, device 102, or any of servers 104, may determine a second environmental parameter, e.g., an altitude of the user or device 102.

At block 410, any of device 102 and servers 104 may determine a third environmental parameter, e.g., a weather associated with the user or device 102.

At block 412, device 102 may display, by way of display 112, outputs such as a replacement rate (e.g., for sodium, potassium, carbohydrates, and fluid) in response to the body parameters and environmental parameters.

After block 412, the process 400 ends.

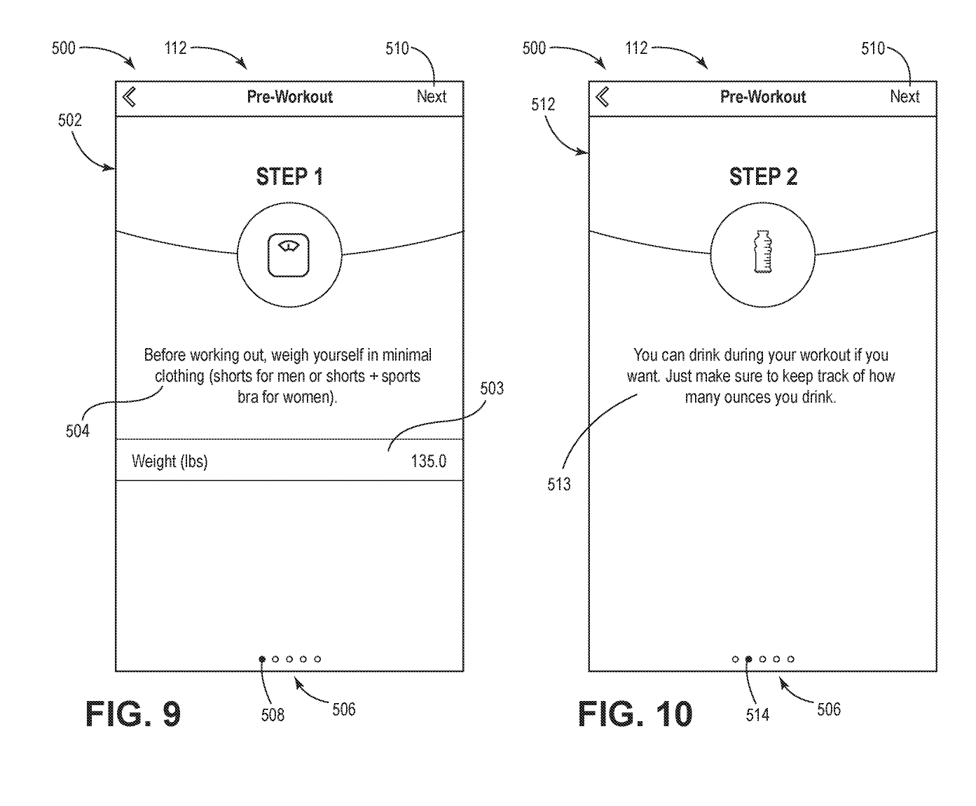

FIGS. 9-11 illustrate system 500. System 500 may include all or any portion of the systems herein, e.g., systems 100, 200, 300, and 400. System 500 may take many different forms and include multiple and/or alternate hardware components and implementations. While an exemplary system is shown in the figures, the exemplary components illustrated are not intended to be limiting. Indeed, additional or alternative components and/or implementations may be used.

For example, system 500 may include processor 106, memory 108, display 112, transceiver 116, sensors 103 and 114, servers 104, and database 122. Memory 108 may include program 110 communicatively connected to processor 106 that when executed provides the operations herein. Processor 106 may provide operations to receive a historical excretion rate of a user by way of display 112, memory 108 or database 122, receive a body parameter of a user by way of display 112, memory 108 or database 122, receive a sensor output (e.g., body sensor output) from sensors 103 (e.g., body sensors), and generate a projected excretion rate of the user based on the historical excretion rate, the body parameter, and the sensor output. System 500 may display, by the display 112, body performance information of the user. Methods may include receiving a historical excretion rate of a user, receiving a body parameter of the user, receiving sensor outputs, generating a projected excretion rate of the user based on the historical excretion rate, the body parameter, and the sensor outputs, and displaying a body performance output of the user based on the projected excretion rate.

As shown in FIG. 9, system 500 may include display 112 having, for example, screen 502 for pre-activity operations to be displayed prior to the activity or workout. Screen 502 may indicate “Pre-Workout” and “STEP 1.” Screen 502 may include input area 503, instruction area 504 and indicators 506. Input area 503 may include a request for user inputs, e.g., to enter body parameters of the user. For example, input area 503 may include a request for user weight. Instruction area 504 may include a predefined level of clothing (e.g., minimal clothing) and gender specific guidance (e.g., applicable to only men or only women). System 500 may include indicators 506, e.g., one or a series of lighted indicators. Indicators 506 may include a first indicator 508, e.g., corresponding to screen 502 or input area 503 and instructions area 504. System 500 may also be responsive to user inputs. For example, system 500 may advance to the next screen in response to the user swiping on a touchscreen or pressing or otherwise activating next button 510, which advances display 112 to screen 512 of FIG. 10.

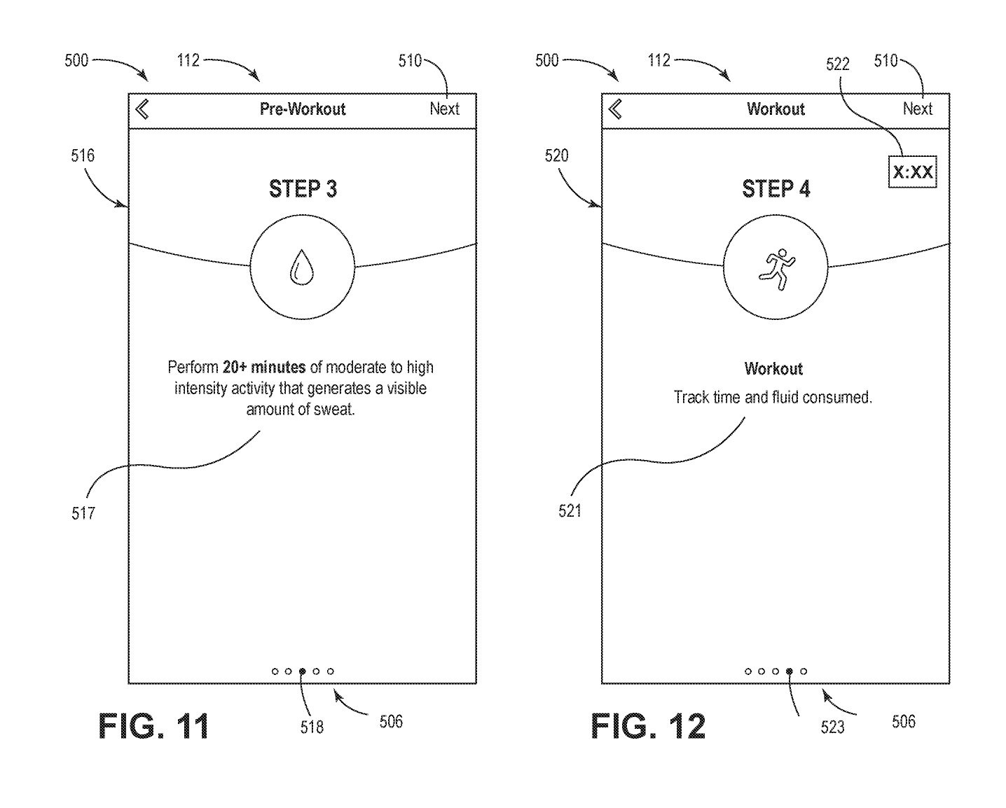

FIG. 10 illustrates system 500 that may include display 112 having screen 512 for pre-activity operations, for example, to be displayed prior to the activity or workout. Screen 512 may indicate “Pre-Workout” and “STEP 2.” Instructions area 512 may display user instructions indicating that the user may consume replenishments (e.g., a drink or food). Instructions area 512 may be displayed during the activity. System 500 may track and store a replenishment amount, e.g., a weight or volume associated with consumed replenishments. The replenishment amount may be received by user inputs by way of display 112 or from one or more of sensors 103 or 114. Indicators 506 may correspond to screen 512 or instructions area 513. Indicators 506 may include a second indicator 514, which may be illuminated. System 500 may advance to the next screen as described above, which advances the display 112 to screen 516 of FIG. 11.

FIG. 11 illustrates system 500 that may include display 112 having screen 516 for pre-activity operations, for example, to be displayed prior to the activity or workout. Screen 516 may include “Pre-Workout” and “STEP 3.” Screen 516 may include instruction area 517. Instruction area 517 may instruct the user to perform an activity having a predefined time (e.g., about 20 or 45 minutes), a predefined intensity or intensity range (e.g., low, moderate, high intensity, or a range therebetween), and a predefined target (e.g., generating a visible amount of sweat or a low, modest or heavy level of breathing). The instruction area 517 may be generated based on user inputs, e.g., selections related to an activity such as a particular sport, method, or difficulty. Thus, the predefined target may be generated and displayed in response to a selected activity or sport (e.g., running or bicycling). That is, in response to user inputs received as part of a predefined workout, screen 516 displays a recommended workout routine that corresponds with, for example, the user inputs and body performance information. Screen 516 may include indicators 506 corresponding to screen 516. Indicators 506 may include a third indicator 518, which may be illuminated. System 500 may advance to the next screen in response to the user swiping on a touchscreen or pressing or otherwise activating the “next” button 510, which advances the display 112 to screen 520 of FIG. 12.

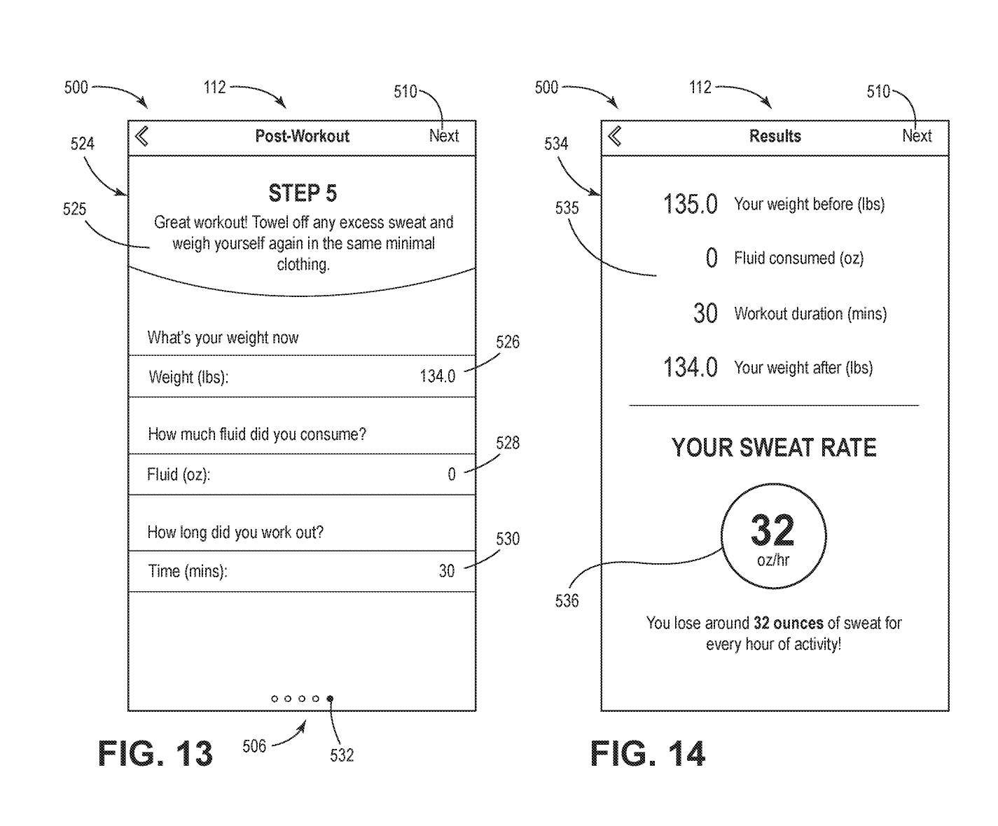

As shown in FIG. 12, system 500 may include display 112 having screen 520 for in-activity operations, e.g., during the activity or workout. Screen 520 may indicate “Workout” and “STEP 4.” Screen 520 may include indication 521 for the user to proceed with the activity or workout. Indication 521 may include, and system 500 may track and store, an activity time and fluid consumption. Screen 520 may also include a synopsis of user inputs (e.g., selected sport or exercise). Screen 520 may be periodically or continuously displayed as a reminder of the user inputs and pre-workout details. In addition, screen 520 may include a timer or clock 522 so that time may be displayed during the workout. The timer 522 may be activated automatically by processor 106 or in response to user inputs by way of the display 112. The workout time may be stored in memory 108 or database 122. Indicators 506 may include a fourth indicator 523. System 500 may advance to the next screen in response to the user swiping on a touchscreen or pressing or otherwise activating the “next” button 510, which advances the display 112 to screen 524 of FIG. 13.

FIG. 13 illustrates system 500 that may include display 112 having screen 524 for post-activity operations. Screen 524 may indicated “Post-Workout” and “STEP 5.” Screen 524 may include instruction area 525, weight input 526, consumption input 528, time input 530, and indicators 506. For example, a user weight may be received by way of a user input in weight input 526, an amount of fluid or food consumed during the workout may be received by way of a user input in consumption input 528, and an activity time (e.g., workout time) may be received by way of a user input in time input 530. The activity time may also be automatically generated by way an internal clock associated with processor 106 or by way of a separate stopwatch, wrist watch, or wall clock. Indicators 506 may include a fifth indicator 532 corresponding to instruction area 525, weight input 524, consumption input 528, and time input 530. System 500 may advance to the next screen in response to the user swiping on a touchscreen or pressing or otherwise activating the “next” button 510, which advances the screen display to screen 534 of FIG. 14.

As shown in FIG. 14, system 500 may include display 112 having screen 534 for displaying body performance information, e.g., following an activity or workout. Screen 534 may include inputs area 535 and results area 536. Inputs area 535 may include the user inputs that were previously entered in screen 524 or user inputs from memory 108 or database 122. For example, inputs area 535 may include a user weight (e.g., from input area 503), a consumption amount (e.g., from consumption input 528), a workout or activity duration (e.g., time input 530), and a user weight (e.g., time input 530). Results area 536 may include body performance information (e.g., a sweat rate). The sweat rate may be displayed in units of a mass of sweat per unit time. For example, the mass of sweat may be in units of ounces (oz), grams or any other unit of mass. Further, the unit time is displayed as per hour, but it is contemplated that any unit of time may be used, such as minutes or seconds. And, any combination thereof may also be displayed, such as milligrams/second, grams/minute, and the like. As such, results area 536 may display the sweat rate based on, for example, a measured change in weight, time of workout, and taking into account any fluid input. In addition, additional body waste may be expelled during, for instance, an extended workout. Thus, the systems herein may measure an amount of urine or other waste that may be expelled during a workout, which may be estimated and entered either by adjusting consumption input 528, or by having a separate area on display 112 for such entry.

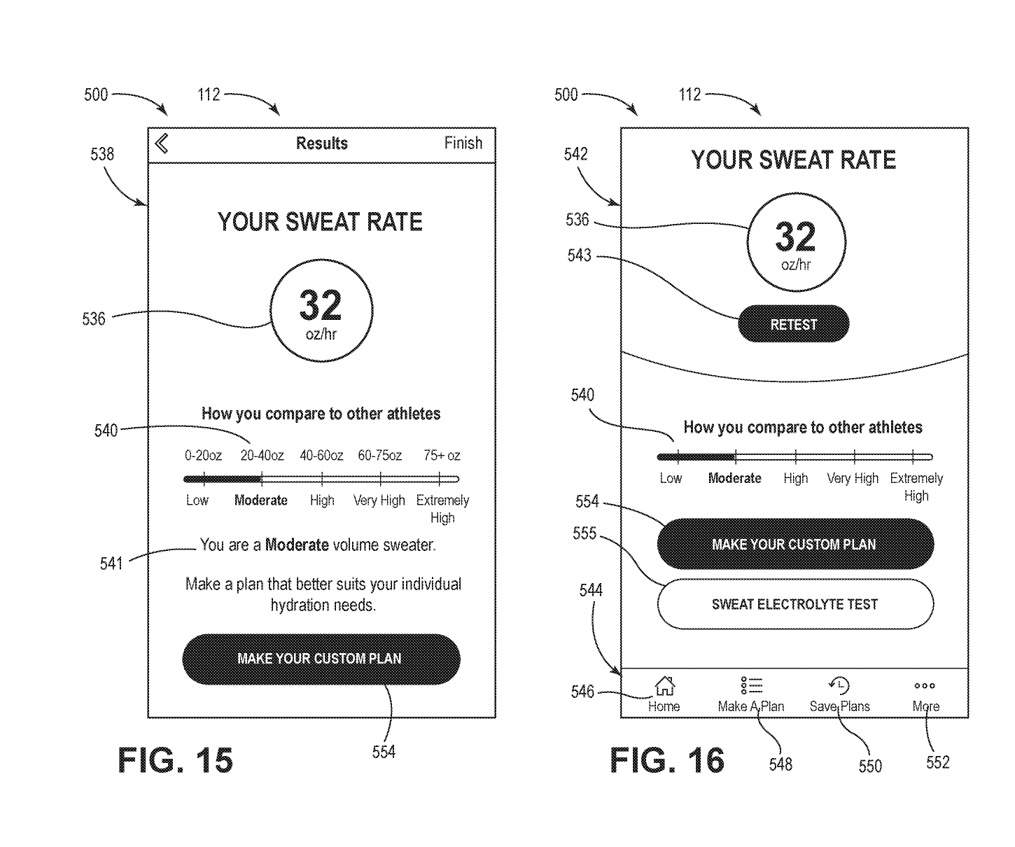

As shown in FIG. 15, system 500 may include display 112 having screen 538 for displaying body performance information. Screen 538 may include body performance information in results area 536 (e.g., including a sweat rate) and comparison area 540. The results area 536 may include the sweat rate of the user of device 102 or sensors 103. Comparison area 540 may include a comparison between the sweat rate in results area 536 (e.g., of the user of device 102 or sensors 103) and one or a plurality of sweat rates of other users by way of devices 102, sensors 103, memory 108, database 122, or a combination thereof. The comparison in comparison area 540 may include an indication of a relatively low, moderate, high, very high, and extremely high excretion or sweat rate compared to other users, and their corresponding historical excretion rates that may be stored in database 122 or memory 108. Thus, body performance information of users of other devices 102 or sensors 103 may be obtained and utilized for comparison purposes.

As such, the systems and users as described herein can learn from one another so that body performance information (e.g., sweat rate trends) may be extrapolated for body performance information of other devices and users (e.g., based on other bodily or environmental conditions, exercise types, and the like). As one example, for the given set of conditions in the previously explained example, results area 536 may include a sweat rate as in this example was determined to be 32 oz/hour, which may be displayed as a “moderate” sweat rate, for the given conditions, compared to other athletes. As such, as further data is obtained for the user, the user and device 500 may normalize, and develop a historical excretion or sweat rate, and display a sweat rate that may be compared to other activities. For instance, in the above example, 30 minutes of exercise resulted in a loss of 1 pound (lb) of sweat, for a displayed sweat rate of 2 lb, or 32 oz, per hour. In comparison and against other athletes for the same conditions, other athletes may have a “high” rate of 40-60 oz/hour, or a “very high” rate of 60-75 oz/hour, as examples displayed.

FIG. 16 displays system 500 that may include display 112 having screen 542 with retesting options that may additionally or alternatively be available. Screen 524 may include results area 536, retest selection 543, custom plan selection 554, and test selection 555. Results area 536 may include body performance information such as a sweat rate. Retest selection 543 may be selected and restart system 500 at any of the preceding screens or a predefined restart screen. Test selection 55 may run a sweat electrolyte test to generate replacement rates as discussed above. More specifically, test selection 555 may determine and estimate an amount of electrolytes to be replaced, as a result of the previous exercise routine, based on such factors as the assumed consumption of sodium and/or potassium. The test selection 555 may also include an option for entering electrolyte information, which may be directly measured using a separate electrolyte testing device, in which electrolytes are measured directly in a blood sample, as an example. System 500 also displays, as an example, a set of options via a menu 544, to include displaying an option to move to home 546, make a plan 548, and save a plan 550. More options 552 may also be available, displaying options available to move to any of the other screens herein. In fact, “more” options of FIGS. 2-4 (and as illustrated as element 222 in FIG. 2), may allow for moving back and forth, to allow setting up a new plan or correcting data entered in an existing plan, without loss of other settings.

A feature that may be available in both options of FIGS. 15 and 16 may be to make a custom plan, displayed as element 554. A custom plan may be prepared based on data obtained for an individual for different exercise routines, and/or based on comparison of the individual against other athletes.

For instance, as indicated in the example and as displayed in FIGS. 15-16, the user experienced a loss of 32 oz/hour (oz/hr) for all the previously defined exercise conditions. The measured sweat rate indicated that, for similar conditions, the individual sweats at a “moderate” rate in comparison to other athletes. As such, and as more data is obtained for this user, a trend of sweat rates may be determined, displayed and confirmed over time by system 500. That is, over several tests the user may regularly and repeatedly fall in the “moderate” sweat rate range, thus providing an opportunity to extrapolate and/or predict how this user will perform not only for the given exercise, but for other exercise activities for the user as well.

As another example, a user may not yet have performed any sweat rate analysis for a given exercise. However, if the user routinely falls into the “moderate” category, then data for other exercise routines may be applied to this user, and plans may be constructed based on measurements obtained for a population of users for a given set of conditions. And, if the user has already performed multiple exercise routines for a given exercise, then that data may be utilized for constructing an exercise plan, as well.

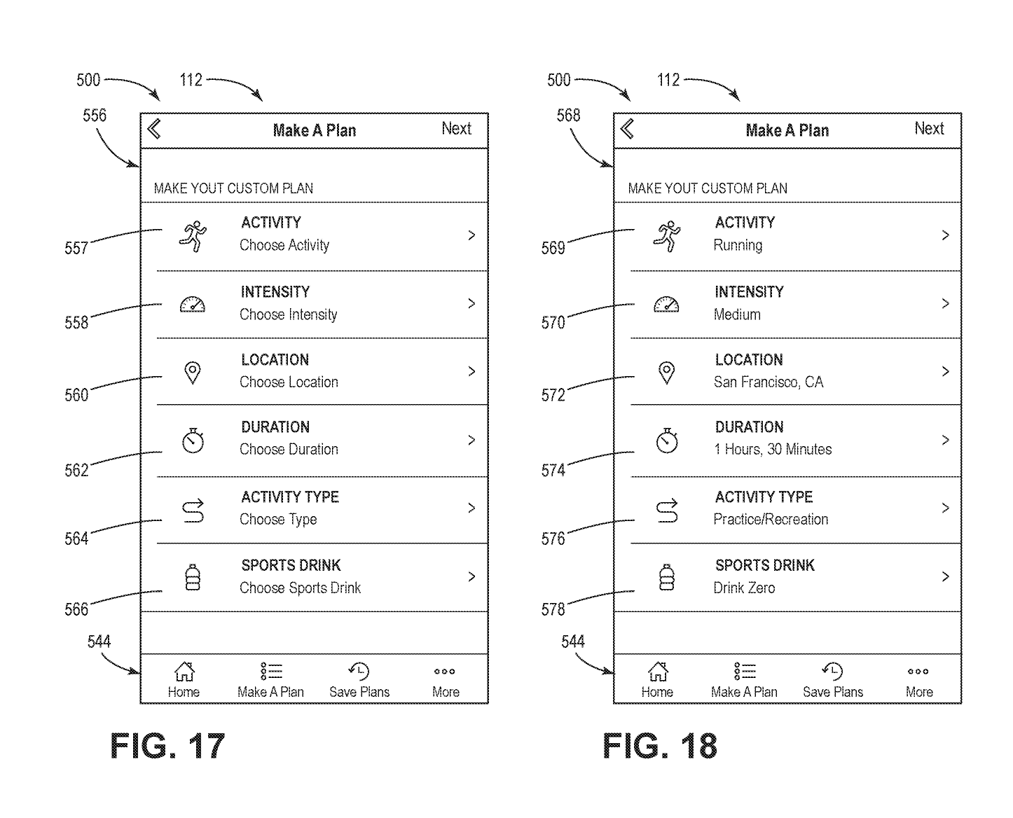

As shown in FIG. 17, system 500 may include display 112 having screen 556 to generate an activity or workout plan. Screen 556 may be configured to receive user inputs includes an activity selection 557, an intensity selection 558, a location selection 560 (e.g., geographic location by way of GPS of device 102), a duration selection 562 (e.g., time duration of activity), an activity type selection 562, and a replenishment selection 566 (e.g., drink or food). Screen 556 may also include menu 544.

FIG. 18 illustrates system 500 that may include display 112 having screen 568 that displays the selected user inputs. For example, screen 568 may include a selected activity 569 (e.g., running or walking), a selected intensity 570 (e.g., medium, low, or high), a selected location 572 (e.g., San Francisco, Calif.), a selected duration (e.g., one hour and thirty minutes), a selected activity type 576 (e.g., “practice/recreation”), and a selected replenishment 578 (e.g., sports drink). Screen 568 may also include menu 544.

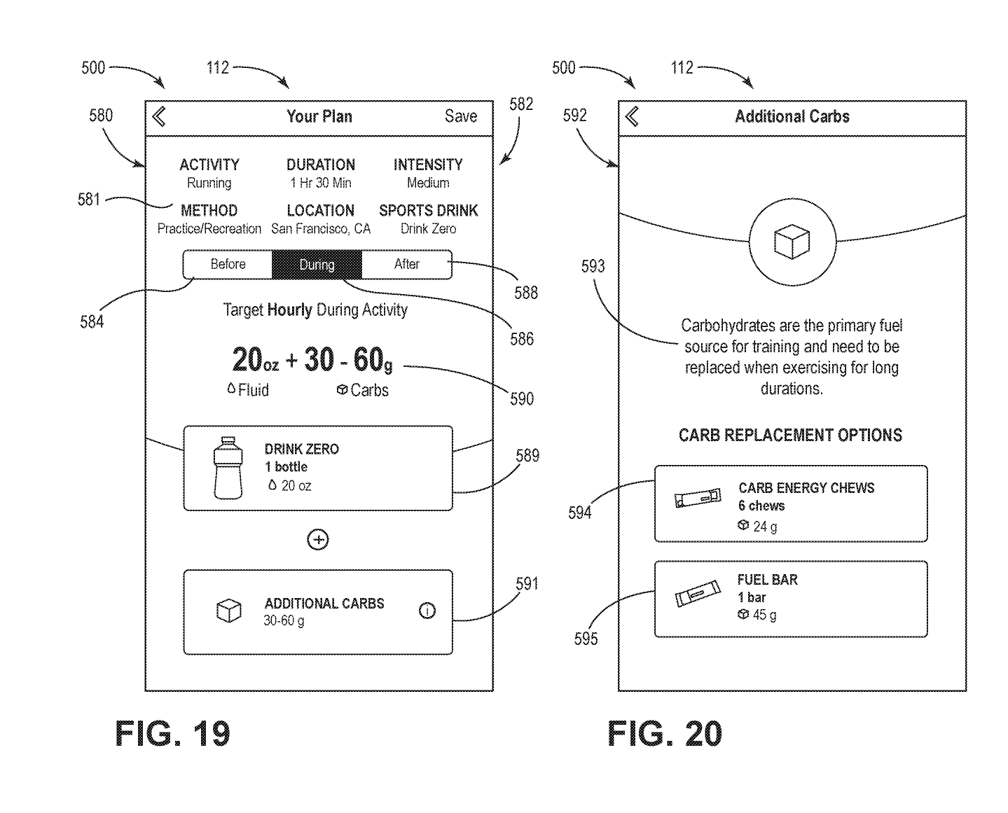

FIG. 19 illustrates system 500 that may include display 112 having a plan displayed as a summary. System 500 may include screen 580 having display elements of a plan 581 including a summary of user inputs, e.g., an activity, duration, intensity, method, location, and replenishment. The method selection may indicate “practice/recreation” as the selected activity type 576 previously received on screen 568 as a user input, for example. System 500 displays options for user selection, such as before 584, during 586, and after 588 a planned activity. Each of the displayed items of before 584, during 586, and after 588 display a corresponding instruction to the user, received from user inputs of FIG. 17. For example, fluid intake (20 oz) and range of carbs (30-60 g) 590 are displayed as target hourly actions for during the planned activity, which are obtained using processor 106 from any one of database 122 and memory 108. “During” display 586 is darkened, indicating target fluid intake of 20 oz, and target range of carbs as 30-60 g, to be taken during the planned activity. Similarly, target drink and carb levels may be displayed before and after exercise, by from inputs before 584 or after 588. As with the other screens, system 500 may advance to the next screen in response to the user wiping on a touchscreen or otherwise activating a “next” button (not shown). A results area 589 may indicate one of the indicated items in display 586, providing details corresponding to the instruction (e.g., for the user to drink one bottle of “DRINK ZERO”), obtained from any one of database 122 and memory 108. A results area 591 may indicate one of the indicated items in display 586, providing details corresponding to the instruction (e.g., for the user to consume additional carbs). Results areas 589 and 591 may obtain data for display from database 122.

FIG. 20 illustrates system 500 that may include display 112 having screen 592 for displaying replenishment options (e.g., options for carbohydrate replenishment). Screen 592 may include instructions 593, replenishment option 594, and replenishment option 595. Instruction 593 may provide information regarding carbohydrate replacement (e.g., the purpose and benefits thereof). Instruction 593 may be received from memory 108 or database 122. Replenishment option 594 may include, for example, the option of “CARB ENERGY CHEWS” having 24 g/6 chews. Replenishment option 595 may include, for example, the option “FUEL BAR” having 45 g/bar 594.

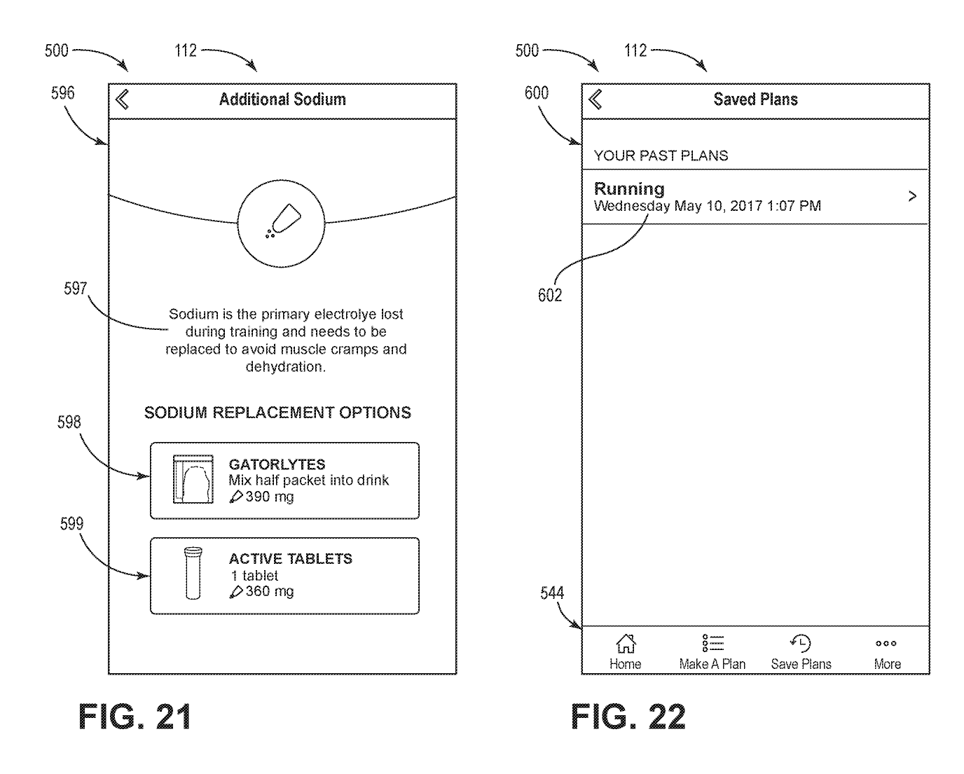

FIG. 21 illustrates system 500 having display 112 with screen 596 for displaying replenishment options (e.g., options for sodium replenishment). Screen 596 may include instructions 597, replenishment option 598, and replenishment option 599. Instruction 597 may include information regarding sodium replacement (e.g., the purpose and benefits thereof). Replenishment option 598 may include and describe, for example, sodium content in “GATORLYTES” having 390 mg of sodium. Replenishment option 599 may include and describe, for example, “ACTIVE TABLETS” having 360 mg of sodium. User options 598 and 599 may be provided by system 500 from any one of database 122 and memory 108. Further, not only carbs and sodium, but other items such as electrolytes, including potassium, may also be displayed as target replacement or replenishment options for the user.

FIG. 22 displays system 500 that may include display 112 that having screen 600 for displaying past plans. One exemplary past plan 602 is displayed, and past plan 602 may thereby be selected, accessed via system 500 from database 122, or from memory 108 of user profile server 104a, testing lab server 104b, healthcare server 104c, or third-party server 104d. Selection of past plan 602 may thereby access data from any one of database 122 and memory 108 for display.

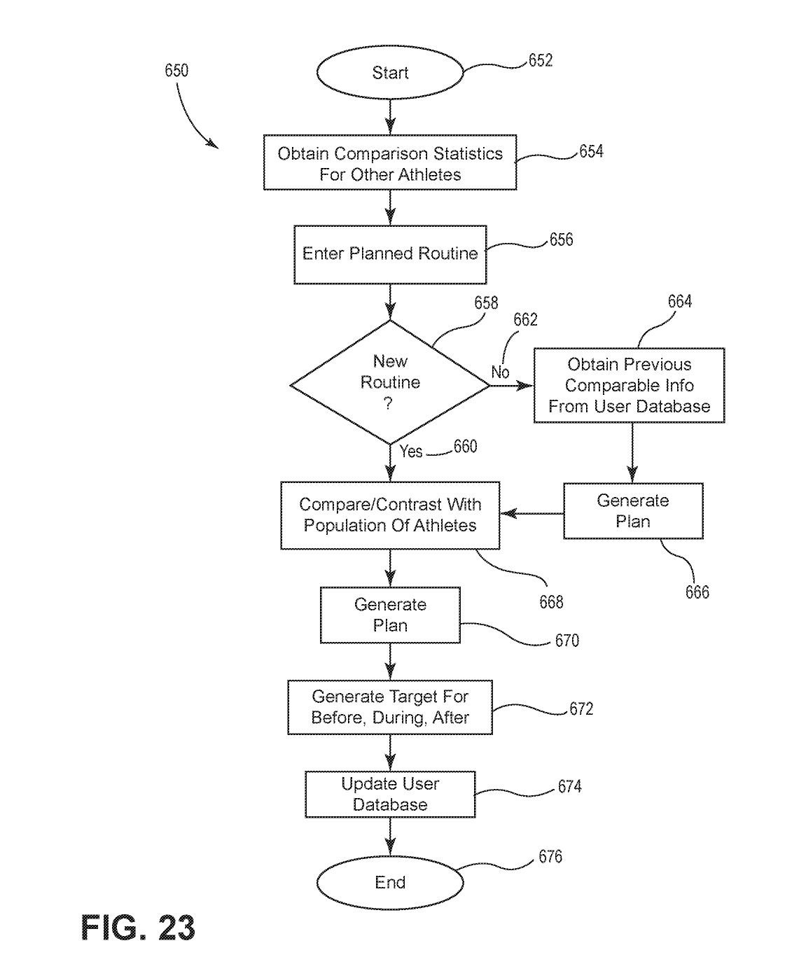

FIG. 23 illustrates an exemplary process 650. Process 650 may take many different forms and include multiple and/or alternate components and implementations. While an exemplary process is shown, the exemplary components illustrated are not intended to be limiting. Indeed, additional or alternative components and/or implementations may be used.

At oval 652, process 650 may start as a new process or may be used as alternative or in addition to other processes (e.g., process 400).

At block 654, processor 106, by way of display 112 and transceiver 116, may receive body performance information. The body performance information may include comparison statistics for other users (e.g., athletes). The body performance information may be received from database 122 or memory 108 of device 102 or servers 104.

At block 656, processor 106, by way of display 112, may receive user inputs including a planned routine for an activity or workout of a user.

At block 658, processor 106 may determine whether a user is performing a new routine based on the planned routine. If processor 106 determines not to be a new routine 662, then the process may proceed to block 664.

At block 664, processor 106 may receive body performance information from memory 108 or database 122. For example, the body performance information may include previous and comparable information, e.g., stored in a user profile server of server 104a. As a user performs exercise or athletic routines, database 122 or memory 108 may be updated for the user.

At block 666, processor 106 may generate a plan as described herein. If processor 106 determines to be a new routine at decision 660, then device 102, any of servers 104, or system 500 determines that the planned routine at block 666 may be based off of statistics derived solely from other athletes. If the processor 106 determines that user has already been characterized for the planned (or other) athletic activities, processor 106 may receive body performance information from memory 108 or database 122 e.g., to determine the user trends in sweat rate.

At block 670, processor 106 may associate and define the sweat rate as a normalizing factor for the plan. More specifically, based on the sweat rate of the user, processor 106 determines that such information may be used to normalize against a one or a plurality of other users. In the above example, such as in FIGS. 15 and 16, system 500 determined the user to have a moderate sweat rate, illustrated in comparison area 540. Thus, when processor 106 generates a new plan (whether the user has yet performed this type of routine or not), and based on whether processor 106 has determined the user to have a moderate sweat rate compared to other users, then device 102, any of servers 104, or system 500 may access a pool of user data from database 122 or memory 108 of device 102 or servers 104a-d. Processor 106 may thereby apply the body performance information of other users to predict replenishment amounts (such as water, electrolyte, and/or carbohydrate requirements) based on the experiences of the other users, by comparing and contrasting in the user to the other users, as in block 668. As such, device 102, any of servers 104, or system 500 may use the sweat rate of the user as an overall normalizing factor to generate a plan, at block 670.

In response to processor 106 generating the plan at block 670, replenishment amounts (e.g., for water, carbohydrates, and electrolytes) are generated by processor 106 at block 672. The generated targets may pertain to periods before, during, and after any exercise routine is carried out, as shown on display 580 of FIG. 19. Further, body performance information of a device 102, any of servers 104, or system 500 may be compared by processor 106 to body performance information of one or a plurality of other users as discussed in block 668.

For instance, in one example a user may reside in Denver, Colo., having a certain geographical elevation and other environmental aspects that may affect athletic performance. During a trip for vacation or for business, the user may travel to San Francisco, Calif. Thus, device 102, any of servers 104, or system 500 may have established substantially all personal data for the Denver location based off of user locations. Given that data, device 102, any of servers 104, or system 500 may determine the user to have a moderate sweat rate against other users, measured against user information from database 122 having other users for conditions in Denver. Device 102, any of servers 104, or system 500 may then compare or contrast against a pool of user data from database 122 as may be obtained in the San Francisco area, when system 500 determines via location determination technology to be in the San Francisco area. Thus, a user having a moderate sweat rate in Denver may be normalized using device 102, any of servers 104, or system 500 to a population in San Francisco, and although the actual and observed sweat rate may be different between the two locations, as a user who experiences a moderate sweat rate, the targets may be extrapolated by system 500 using the population data from San Francisco.

At block 674, processor 106, by way of transceiver 116, updates database 122 or memory 108 of servers 104a-d.

At oval 676, process 652 ends.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A system comprising:
a user interface;
a processor and a memory having a program communicatively connected to the processor, the processor providing operations including:
receive a historical excretion rate, a body parameter, and a sensor output associated with a user;
generate a projected excretion rate based on the historical excretion rate, the body parameter, and the sensor output; and
display, by the user interface, a body performance output of the user based on the projected excretion rate.

2. The system of claim 1, wherein the body performance output includes a replacement rate.

3. The system of claim 2, wherein the replacement rate includes at least one of a sodium replacement rate, a potassium replacement rate, a carbohydrate replacement rate, and a fluid replacement rate.

4. The system of claim 1, the operations further including at least one of:
receive, by way of the user interface, the historical excretion rates from the user; and
receive, by way of a scanner, the historical excretion rate from textual information of a sweat report.

5. The system of claim 1, the operations further including:
receive, by way of a transceiver in communication with a body sensor, the sensor output.

6. The system of claim 1, wherein the generated projected excretion rate is based an environmental parameter including at least one of a user location, an altitude, and a weather.

7. The system of claim 1, wherein the projected excretion rate is generated based on a user location received by way of at least one of the user interface, a global positioning system, and a network.

8. A non-transitory computer-readable medium tangibly embodying computer-executable instructions of a program being executable by a processor of a computing device to provide operations comprising:
receive a historical excretion rate, a body parameter, and a sensor output of a user;
generate a projected excretion rate of the user based on the historical excretion rate, the body parameter, and the sensor output; and
display a body performance output of the user based on the projected excretion rate.

9. The medium of claim 8, wherein the body performance output includes a replacement rate.

10. The medium of claim 9, wherein the replacement rate includes at least one of a sodium replacement rate, a potassium replacement rate, a carbohydrate replacement rate, and a fluid replacement rate.

11. The medium of claim 8, the operations further including at least one of:
receive, by way of the user interface, the historical excretion rates from the user; and
receive, by way of a scanner, the historical excretion rate from textual information of a sweat report.

12. The medium of claim 8, the operations further including:
receive, by way of a transceiver in communication with a body sensor, the sensor output.

13. The medium of claim 8, wherein the generated projected excretion rate is based an environmental parameter including at least one of a user location, an altitude, and a weather.

14. The medium of claim 8, wherein the projected excretion rate is generated based on a user location received by way of at least one of the user interface, a global positioning system, and a network.

15. A method comprising:
receiving a historical excretion rate, a body parameter, and a sensor output associated with a user;
generating a projected excretion rate of the user based on the historical excretion rate, the body parameter, and the sensor output; and
displaying a body performance output of the user based on the projected excretion rate.

16. The method of claim 15, wherein displaying the body performance output includes displaying a replacement rate.

17. The method of claim 16, wherein displaying the replacement rate includes displaying at least one of a sodium replacement rate, a potassium replacement rate, a carbohydrate replacement rate, and a fluid replacement rate.

18. The method of claim 15, further comprising at least one of:
receiving, by way of a user interface, the historical excretion rates from the user; and
receiving, by way of a scanner, the historical excretion rate from textual information of a sweat report.

19. The method of claim 15, further comprising:
receiving, by way of a transceiver in communication with a body sensor, the sensor output.

20. The method of claim 15, wherein generating the projected excretion rate is based an environmental parameter including at least one of a user location, an altitude, and a weather, and wherein generating the projected excretion rate is based on a user location received by way of at least one of the user interface, a global positioning system, and a network.
